(19) United States
(12) Patent Application Publication
(10) Pub. No.: US 2025/0017955 A1
(43) Pub. Date: Jan. 16, 2025

(54) BORONATED HYDROGELS AND METHODS OF MAKING AND USING THE SAME

(71) Applicant: Boston Scientific Scimed, Inc., Maple Grove, MN (US)

(72) Inventors: Yen-Hao Hsu, Shrewsbury, MA (US); Joseph Thomas Delaney, JR., Minneapolis, MN (US); Cristian Parisi, Boston, MA (US)

(73) Assignee: Boston Scientific Scimed, Inc., Maple Grove, MN (US)

(21) Appl. No.: 18/761,777

(22) Filed: Jul. 2, 2024

Related U.S. Application Data
(60) Provisional application No. 63/525,233, filed on Jul. 6, 2023.

(51) Int. Cl. A61K 31/738; C08K 3/011

(52) U.S. Cl. CPC A61K 31/738; C08K 3/011

(57) ABSTRACT

This disclosure pertains to boronated hydrogel compositions, which comprise a boronated polysaccharide compound that comprises a plurality of boronated moieties covalently linked to a carboxylic-acid-containing polysaccharide along a backbone of the carboxylic-acid-containing polysaccharide. The disclosure also pertains to kits that comprise a reservoir, a boronated hydrogel composition comprising a boronated polysaccharide compound that comprises a plurality of boronated moieties covalently linked to a carboxylic-acid-containing polysaccharide along a backbone of the carboxylic-acid-containing polysaccharide disposed in the reservoir, and a device for administering the boronated hydrogel composition to a subject. The disclosure further pertains to methods of treatment that comprise applying or injecting a boronated hydrogel composition comprising a boronated polysaccharide compound that comprises a plurality of boronated moieties covalently linked to a carboxylic-acid-containing polysaccharide along a backbone of the carboxylic-acid-containing polysaccharide onto or into target cells of a subject and delivering neutron beam radiation to the target cells.

### CROSS REFERENCE TO RELATED APPLICATIONS

This application claims the benefit of U.S. Provisional Patent Application Ser. No. 63/525,233 filed on Jul. 6, 2023, the disclosure of which is incorporated herein by reference.

### FIELD

The present disclosure relates to boronated hydrogels and to methods of making and using such hydrogels, among other aspects. The boronated hydrogels of the present disclosure are useful in biomedical applications, including boron neutron capture therapy.

### BACKGROUND

Boron neutron capture therapy (BNCT) is a promising and efficient tool whereby cancers are treated by selectively concentrating boron-compounds close to or in tumor cells and then delivering neutron beam radiation for cancer therapy. Boron neutron capture therapy utilizes boronated agents to preferentially deliver boron-10 atoms to tumors. After undergoing irradiation with neutrons, boron-10 yields lithium-7 and an alpha particle. The alpha particle has a short range, therefore preferentially treats tumor tissues while sparing more distant healthy tissues. More specifically, boron neutron capture therapy is based on nuclear capture and fission that follows irradiation of nonradioactive boron-10 with low thermal neutrons which leads to the production of an alpha particle and a recoiling lithium-7 particle (¹⁰B+¹n→[¹¹B]*→⁴He₂ (α)+7Li₃+2.38 MeV). Alpha particles are a form of high linear energy transfer (LET) particles that deposit their energy over <10 μm. For further information, see, e.g., K. Nedunchezhian. et al., Boron neutron capture therapy-a literature review, Journal of clinical and diagnostic research: JCDR, 10 (12), ZE01 (2016) and T. D. Malouff et al., Boron neutron capture therapy: A review of clinical applications. Frontiers in oncology, 11, 601820 (2021).

Non-animal stabilized hyaluronic acid (NASHA) is a hyaluronic acid preparation that is produced wholly from non-animal sources and is used to form hydrogels that are suitable for injections and as fillers, with good biocompatibility in several applications. One hyaluronic acid-based hydrogel, available as Barrigel®, is used to minimize radiation-associated side effects during prostate cancer treatment by creating the space in between the prostate and rectum.

There is a need in the biomedical arts for hydrogels that are boronated, rendering them potentially useful for boron neutron capture therapy, for methods of making and using such boronated hydrogels, and for systems for forming such boronated hydrogels.

### SUMMARY

In some aspects, the present disclosure pertains to boronated hydrogel compositions that comprise a boronated polysaccharide compound that comprises a plurality of boronated moieties covalently linked to a carboxylic-acid-containing polysaccharide along a backbone of the carboxylic-acid-containing polysaccharide.

In some embodiments, the boronated moieties are covalently linked to the carboxylic-acid-containing polysaccharide through amide or thioester bonds.

In some embodiments, which can be used in conjunction with the above aspects and embodiments, the carboxylic-acid-containing polysaccharide comprises one or more uronic acid species selected from galacturonic acid, glucuronic acid, and iduronic acid.

In some embodiments, which can be used in conjunction with the above aspects and embodiments, the carboxylic-acid-containing polysaccharide is selected from hyaluronic acid, alginic acid, pectin, agaropectin, carrageenan, gellan gum, gum arabic, guar gum, xanthan gum, and carboxymethyl cellulose.

In some embodiments, which can be used in conjunction with the above aspects and embodiments, the boronated moieties are selected from dioxaborolane moieties and dodecaborate moieties.

In some embodiments, which can be used in conjunction with the above aspects and embodiments, the boronated moieties comprise residues of primary-amine-substituted boronated compounds.

In some embodiments, which can be used in conjunction with the above aspects and embodiments, the boronated moieties comprise residues of aminoalkyl dioxaborolane compounds.

In some embodiments, which can be used in conjunction with the above aspects and embodiments, the boronated moieties comprise residues of aminoalkyl phenyl dioxaborolane compounds.

In some embodiments, which can be used in conjunction with the above aspects and embodiments, the boronated moieties comprise residues of thiol-functionalized dodecaborate compounds.

In some embodiments, which can be used in conjunction with the above aspects and embodiments, the boronated polysaccharide is covalently crosslinked.

In some embodiments, which can be used in conjunction with the above aspects and embodiments, the boronated polysaccharide is ionically crosslinked by multivalent cations.

In some aspects, the present disclosure pertains to kits that comprise a reservoir, a boronated hydrogel composition in accordance with any of the above aspects and embodiments disposed in the reservoir, and a device for administering the boronated hydrogel composition to a subject. In some of these embodiments, the reservoir is a syringe barrel.

In some aspects, the present disclosure pertains to methods of treatment that comprise applying or injecting a boronated hydrogel composition in accordance with any of the above aspects and embodiments onto or into target cells of a subject and delivering neutron beam radiation to the target cells. In some of these embodiments, the target cells are surface cancer cells.

The above and other aspects, embodiments, features and benefits of the present disclosure will be readily apparent from the following detailed description.

### BRIEF DESCRIPTION OF THE DRAWINGS

FIG. 1 schematically illustrates a method of making a boronated polysaccharide compound in accordance with an embodiment of the present disclosure.

### DETAILED DESCRIPTION

In the present disclosure, boron-containing molecules are covalently linked to carboxylic-acid-containing polysaccharides to provide hydrogels for cancer therapies, among other potential uses. Utilizing the carboxylic acid groups in the carboxylic-acid-containing polysaccharides provides an opportunity to add additional functionality to the polysaccharides, and the carboxylic-acid-containing polysaccharides can be functionalized with boron-based molecules through the creation of covalent bonds, including amide and thioester bonds. The concentration of the boron moiety can be tuned by varying the density of functionalization of carboxylic acids groups along the polysaccharide backbone.

Carboxylic-acid-containing polysaccharides for use in the present disclosure include any polysaccharide that comprises carboxylic acid groups, including polysaccharides that contain one or more uronic acid species, such as galacturonic acid, glucuronic acid and/or iduronic acid. Particular examples of carboxylic-acid-containing polysaccharides include alginic acid, hyaluronic acid, pectin, agaropectin, carrageenan, gellan gum, gum arabic, guar gum, xanthan gum, and carboxymethyl cellulose moieties. In embodiments where the carboxylic-acid-containing polysaccharide is hyaluronic acid, the carboxylic-acid-containing polysaccharide may be non-animal stabilized hyaluronic acid. In some embodiments, the polysaccharide moiety is derived from a carboxylic-acid-containing polysaccharide having a number average molecular weight ranging from 1 kDa to 8000 kDa, for example ranging anywhere from 1 kDa to 2.5 kDa to 5 kDa to 10 kDa to 25 kDa to 50 kDa to 100 kDa to 250 kDa to 500 kDa to 1000 kDa to 2000 kDa to 8000 kDa (in other words, ranging between any two of the preceding numerical values).

Examples of boronated moieties of the present disclosure include those that comprise one, two, or more boron atoms. In particular embodiments, boronated moieties are selected from organoborane moieties such as dioxaborolane moieties and dodecaborate moieties.

In various embodiments, the boronated compounds of the present disclosure may comprise a carboxylic-acid-containing polysaccharide and a plurality of residues of primary-amine-substituted boronated compounds linked to the carboxylic-acid-containing polysaccharide along a backbone of the carboxylic-acid-containing polysaccharide. The primary-amine-substituted boronated compound residues may be linked along the backbone of the carboxylic-acid-containing polysaccharide through amide linkages.

In various embodiments, a primary amine group of a primary-amine-substituted boronated compound may be reacted with carboxylic acid groups of a carboxylic-acid-containing polysaccharide in an amide coupling reaction to form a boronated polysaccharide. Such an amide coupling reaction may be performed using a suitable coupling reagent, for instance, a carbodiimide coupling reagent such as 1-ethyl-3-(3-dimethylaminopropyl) carbodiimide (EDC) or a salt thereof, such as 1-ethyl-3-(3-dimethylaminopropyl) carbodiimide hydrochloride (EDC·HCl). In various embodiments, between 1% and 100% (for example, ranging from 1% to 2.5% to 5% to 10% to 25% to 50% to 75% to 90% to 95% to 97.5% to 99% to 100%) of the carboxylic acid groups of the carboxylic-acid-containing polysaccharide may be converted to amide groups. The concentration of the boron atoms in the boronated polysaccharide can be tuned by varying the degree of functionalization of the carboxylic acid groups along the polysaccharide backbone.

Examples of primary-amine-substituted boronated compounds include aminoalkyl dioxaborolane compounds (e.g., C₁-C₆-aminoalkyl dioxaborolane compounds) and particularly aminoalkyl phenyl dioxaborolane compounds (e.g., C₁-C₆-aminoalkyl phenyl dioxaborolane compounds). Specific examples include (4-(4,4,5,5-tetramethyl-1,3,2-dioxaborolan-2-yl)phenyl)methanamine,

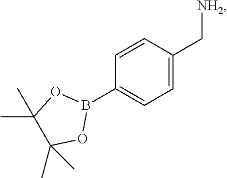 [SMILES: CC1(C)OB(c2ccc(CN)cc2)OC1(C)C]

2-(4-(4,4,5,5-tetramethyl-1,3,2-dioxaborolan-2-yl)phenyl)ethan-1-amine,

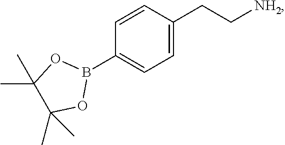 [SMILES: CC1(C)OB(c2ccc(CCN)cc2)OC1(C)C]

2-(4-(4,4,5,5-tetramethyl-1,3,2-dioxaborolan-2-yl)phenyl)ethan-2-amine, 3-(4-(4,4,5,5-tetramethyl-1,3,2-dioxaborolan-2-yl)phenyl)propan-1-amine,

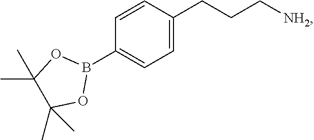 [SMILES: CC1(C)OB(c2ccc(CCCN)cc2)OC1(C)C]

3-(4-(4,4,5,5-tetramethyl-1,3,2-dioxaborolan-2-yl)phenyl)propan-2-amine, 2-((4-(4,4,5,5-tetramethyl-1,3,2-dioxaborolan-2-yl)benzyl)oxy)ethan-1-amine,

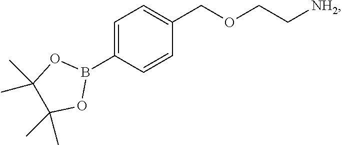 [SMILES: CC1(C)OB(c2ccc(COCCN)cc2)OC1(C)C]

and 4-(4,4,5,5-Tetramethyl-1,3,2-dioxaborolan-2-yl)benzyl (2-aminoethyl)carbamate,

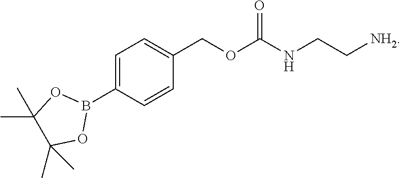 [SMILES: CC1(C)OB(c2ccc(COC(=O)NCCN)cc2)OC1(C)C]

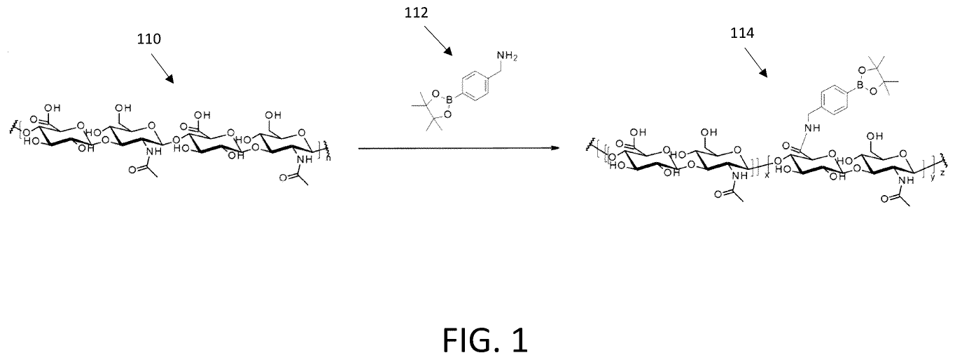

In a particular example shown in FIG. 1, hyaluronic acid is employed as the carboxylic-acid containing polysaccharide and (4-(4,4,5,5-tetramethyl-1,3,2-dioxaborolan-2-yl)phenyl)methanamine (CAS #138500-88-6) is employed as the primary-amine-substituted boronated compound. 1-ethyl-3-(3-dimethylaminopropyl) carbodiimide (EDC) is used as an amide coupling reagent.

In various embodiments, the boronated compounds of the present disclosure may comprise a carboxylic-acid-containing polysaccharide and a plurality of residues of thiol-substituted boronated compounds linked to the carboxylic-acid-containing polysaccharide along a backbone of the carboxylic-acid-containing polysaccharide. The thiol-substituted boronated compound residues may be linked along the backbone of the carboxylic-acid-containing polysaccharide through thioester linkages.

In various embodiments, a thiol group of a thiol-substituted boronated compound may be reacted with carboxylic acid groups of a carboxylic-acid-containing polysaccharide in a thioester coupling reaction to form a boronated polysaccharide. Such a thioester coupling reaction may be performed using a suitable coupling reagent, for instance, using a carbodiimide coupling reagent such as N,N′-dicyclohexylcarbodiimide (DCC) or 1-ethyl-3-(3-dimethylaminopropyl) carbodiimide (EDC). In various embodiments, between 1% and 100% (for example, ranging from 1% to 2.5% to 5% to 10% to 25% to 50% to 75% to 90% to 95% to 97.5% to 99% to 100%) of the carboxylic acid groups of the carboxylic-acid-containing polysaccharide may be converted to thioester groups.

Examples of thiol-substituted boronated compounds include, borocaptate sodium, also known as 1,2,3,4,5,6,7,8,9,10,11-undecahydro-12-mercapto dodecaborate (2-), sodium,

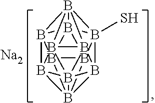

1,2,3,4,5,6,7,8,9,10,11-undecahydro-12-(mercaptomethyl) dodecaborate (2-), sodium, 1,2,3,4,5,6,7,8,9,10,11-undecahydro-12-(2-mercaptoethyl) dodecaborate (2-), sodium, or 1,2,3,4,5,6,7,8,9,10,11-undecahydro-12-(3-mercaptopropyl) dodecaborate (2-), sodium.

Using this and other synthetic procedures, a variety of boronated polysaccharide compounds can be provided which comprise a polysaccharide moiety and a plurality of boronated moieties that are covalently linked along a backbone of the polysaccharide moiety through amide groups or thioester groups.

In various embodiments, boron atoms in the boron-containing molecules of the present disclosure, including the primary-amine-substituted boronated compounds and the thiol-substituted boronated compound boronated compounds of the present disclosure, are enriched with ¹⁰B, as boron has two naturally occurring stable isotopes, ¹¹B (80.1%) and ¹⁰B (19.9%). For example, at least 90%, at least 95%, at least 99% or more of the boron atoms in the boron-containing molecules may be ¹⁰B atoms.

In various embodiments, the boronated polysaccharide compounds of the present disclosure are ionically or covalently crosslinked with one another.

In some embodiments, the polysaccharide moieties of the boronated polysaccharide compounds are covalently crosslinked with one another using a suitable covalent crosslinking agent. Examples of covalent crosslinking agents include multifunctional crosslinking agents having two or more reactive groups, such as divinyl sulfone, crosslinking agents having two or more glycidyl groups (e.g., glycerol diglycidyl ether, 1,4-butanediol diglycidyl ether, ethylene glycol diglycidyl ether, propylene glycol diglycidyl ether, diglycidyl ethers of polyethylene glycol and polypropylene glycol, etc.), crosslinking agents having two or more epoxide groups, crosslinking agents having two or more aldehyde groups, crosslinking agents having two or more isocyanate groups, and crosslinking agents having two or more carboxylic acid groups.

In certain embodiments, the polysaccharide moieties of the boronated polysaccharide compounds are ionically crosslinked with one another using a suitable ionic crosslinking agent. Examples of ionic crosslinking agents include multi-valent cations that can be used to ionically crosslink negatively charged groups of carboxylate-anion-containing polysaccharides (i.e., deprotonated versions of carboxylic-acid-containing polysaccharides). The negatively charged carboxylate anion groups of the carboxylate-anion-containing polysaccharides can form ionic bonds (also referred to as electrostatic bonds) with the multivalent cations, which act to ionically crosslink the carboxylate-anion-containing polysaccharides. Examples of multi-valent cations include Mg²⁺, Ca²⁺, Al³⁺, Fe³⁺, Sr²⁺, Ba²⁺, Bi³⁺, Gd³⁺, Ta⁵⁺, W⁶⁺ and Au⁴⁺, among others, some of which provide radiopacity to the crosslinked product.

In some embodiments, such ionically crosslinked polysaccharides can be formed by first deprotonating a boronated carboxylic-acid-containing polysaccharide such as one of those described above. For example, a boronated carboxylic-acid-containing polysaccharide can be treated with a base such as sodium hydroxide or potassium hydroxide to form a carboxylate-anion-containing polysaccharide salt, preferably a monovalent cationic salt, for example, an alkali metal salt such as a lithium (Li⁺), sodium (Na⁺), potassium (K⁺), rubidium (Rb⁺), cesium (Cs⁺) or francium (Fr⁺) salt. Then the monovalent cations are exchanged with multivalent cations, for example, by mixing an aqueous solution of MgCl₂, CaCl₂), AlCl₃, FeCl₃, SrCl₂, BaCl₂, BiCl₃, GdCl₃, TaCl₅, WCl₆, or AuCl₄ with the boronated carboxylate-anion-containing polysaccharide salt forming a polycluster by coordination.

Crosslinked boronated polysaccharides in accordance with the present disclosure, may vary widely in size, for example, ranging from 10 nm or less to 100 mm or more in largest cross-sectional dimension (e.g., diameter for a sphere, length for a rod, etc.), for example, ranging from 10 nm to 100 nm to 1 μm to 10 μm to 100 μm to 1 mm to 10 mm to 100 mm.

In some embodiments, the crosslinked boronated polysaccharides are in the form of particles. In some embodiments, a boronated polysaccharide compound is first crosslinked and then granulated to form crosslinked boronated polysaccharide particles of suitable size. Granulating may be by any suitable process, for instance by grinding (including cryogrinding), homogenization, crushing, milling, pounding, or the like.

In some embodiments, an emulsion is formed that contains (a) a discontinuous first phase (e.g., an aqueous phase) that contains a boronated polysaccharide dispersed in (b) a continuous second phase (e.g., an oil phase), after which the boronated polysaccharide is crosslinked to form crosslinked boronated polysaccharide particles. A homogenizer or sonicator can be used to reduce the dispersed phase droplet size in order to reduce the final crosslinked boronated polysaccharide particle size.

Sieving or other known techniques can be used to classify and fractionate crosslinked boronated polysaccharide particles according to particle size.

Boronated hydrogel compositions in accordance with the present disclosure may contain additional agents. Examples of additional agents include therapeutic agents. Examples of therapeutic agents include antithrombotic agents, anticoagulant agents, antiplatelet agents, thrombolytic agents, antiproliferative agents, anti-inflammatory agents, hyperplasia inhibiting agents, antibiotics, antimicrobials, analgesics, anesthetics, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists among others.

Examples of additional agents also include colorants such as brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents also include imaging agents. Such imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope ^{99m}Tc, as well as other gamma emitters such as ¹²³I, ¹²⁵I, ¹³¹I, ¹¹¹In, ⁵⁷Co, ¹⁵³Sm, ¹³³Xe, ⁵¹Cr, ^{81m}Kr, ²⁰¹Tl, ⁶⁷Ga, and ⁷⁵Se, among others, (e) positron emitters, such as ¹⁸F, ¹¹C, ¹³N, ¹⁵O, and ⁶⁸Ga, among others, may be employed to yield functionalized radiotracer coatings, (f) radiocontrast agents (beyond any radiopaque crosslinking cations that may be present), for example, particles of tantalum, tungsten, rhenium, niobium, molybdenum, and their alloys, which metallic particles may be spherical or non-spherical, non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®), and (g) contrast agents for use in connection with near-infrared (NIR) imaging, which can be selected to impart near-infrared fluorescence, allowing for deep tissue imaging, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, NIR-sensitive dyes including cyanine dyes, squaraines, phthalocyanines, porphyrin derivatives and borondipyrromethane (BODIPY) analogs, among others.

Boronated hydrogel compositions in accordance with the present disclosure may be provided in a fluid form that is suitable for applying the boronated hydrogel compositions onto tissue (e.g., by spraying, extruding, brushing, etc.) or injecting the boronated hydrogel compositions into tissue. Boronated hydrogel compositions in accordance with the present disclosure may also be provided in a dry form (e.g., powder form) to which a suitable liquid such as water for injection, normal saline, or phosphate buffered saline may be added to form a fluid boronated hydrogel composition suitable for application onto tissue or injection into tissue. Boronated hydrogel compositions in accordance with the present disclosure may be provided in a suitable reservoir, for example, a syringe, vial, or other reservoir.

In various embodiments, kits are provided that include one or more delivery devices for delivering the boronated hydrogel compositions to a subject. Such systems may include one or more of the following: a syringe barrel, which may or may not contain a boronated hydrogel composition as described herein; a vial, which may or may not contain a boronated hydrogel composition as described herein; a needle; a dispenser (e.g., a sprayer); a flexible tube (e.g., adapted to fluidly connect the needle or dispenser to the syringe); and/or an injectable liquid such as water for injection, normal saline or phosphate buffered saline.

The boronated hydrogel compositions described herein can be used for boron neutron capture therapy. In a first step, a boronated hydrogel composition is applied onto or injected into target cells (e.g., cancer cells or other diseased cells). After this step, a period of time may be allowed to elapse to allow the boronated polysaccharide compound to be taken up by the target cells. Subsequently, neutron beam radiation is then delivered to the boronated polysaccharide compound, leading to the transformation of boron-10 atoms into lithium-7 atoms and alpha particles which can kill or slow the growth of the target cells.

Disease sites contemplated for treatment by the present disclosure include lesions, surface-based cancers such as bladder cancers, and interstitial cystitis. Boron neutron capture therapy has also been used to treat a variety of additional disease sites, including glioblastoma multiforme, meningioma, head and neck cancers, lung cancers, breast cancers, hepatocellular carcinoma, sarcomas, cutaneous malignancies, extramammary Paget's disease, recurrent cancers, pediatric cancers, and metastatic disease.

1. A boronated hydrogel composition comprising a boronated polysaccharide compound that comprises a plurality of boronated moieties that are covalently linked to a carboxylic-acid-containing polysaccharide along a backbone of the carboxylic-acid-containing polysaccharide.

2. The boronated hydrogel composition of claim 1, wherein the boronated moieties are covalently linked to the carboxylic-acid-containing polysaccharide through amide or thioester bonds.

3. The boronated hydrogel composition of claim 1, wherein the carboxylic-acid-containing polysaccharide comprises one or more uronic acid species selected from galacturonic acid, glucuronic acid, and iduronic acid.

4. The boronated hydrogel composition of claim 1, wherein the carboxylic-acid-containing polysaccharide is selected from hyaluronic acid, alginic acid, pectin, agaropectin, carrageenan, gellan gum, gum arabic, guar gum, xanthan gum, and carboxymethyl cellulose.

5. The boronated hydrogel composition of claim 1, wherein the boronated moieties are selected from dioxaborolane moieties and dodecaborate moieties.

6. The boronated hydrogel composition of claim 1, wherein the boronated moieties comprise residues of primary-amine-substituted boronated compounds.

7. The boronated hydrogel composition of claim 1, wherein the boronated moieties comprise residues of aminoalkyl dioxaborolane compounds.

8. The boronated hydrogel composition of claim 1, wherein the boronated moieties comprise residues of aminoalkyl phenyl dioxaborolane compounds.

9. The boronated hydrogel composition of claim 1, wherein the boronated moieties comprise residues of thiol-functionalized dodecaborate compounds.

10. The boronated hydrogel composition of claim 1, wherein the boronated polysaccharide is covalently crosslinked.

11. The boronated hydrogel composition of claim 1, wherein the boronated polysaccharide is ionically crosslinked by multivalent cations.

12. A kit comprising (a) a reservoir, (b) a boronated hydrogel composition comprising a boronated polysaccharide compound that comprises a plurality of boronated moieties that are covalently linked to a carboxylic-acid-containing polysaccharide along a backbone of the carboxylic-acid-containing polysaccharide disposed in the reservoir and (c) a device for administering the boronated hydrogel composition to a subject.

13. The kit of claim 12, wherein the reservoir is a syringe barrel.

14. The kit of claim 12, wherein the boronated moieties are covalently linked to the carboxylic-acid-containing polysaccharide through amide or thioester bonds.

15. The kit of claim 12, wherein the boronated moieties are selected from dioxaborolane moieties and dodecaborate moieties.

16. The kit of claim 12, wherein the boronated moieties comprise residues of primary-amine-substituted boronated compounds, residues of aminoalkyl dioxaborolane compounds, residues of aminoalkyl phenyl dioxaborolane compounds or residues of thiol-functionalized dodecaborate compounds.

17. The kit of claim 12, wherein the boronated polysaccharide is covalently crosslinked.

18. The kit of claim 12, wherein the boronated polysaccharide is ionically crosslinked by multivalent cations.

19. A method of treatment comprising (a) applying or injecting a boronated hydrogel composition comprising a boronated polysaccharide compound that comprises a plurality of boronated moieties that are covalently linked to a carboxylic-acid-containing polysaccharide along a backbone of the carboxylic-acid-containing polysaccharide onto or into target cells of a subject and (b) delivering neutron beam radiation to the target cells.

20. The method of claim 19, wherein the target cells are surface cancer cells.
